(19) United States
(12) Patent Application Publication
(10) Pub. No.: US 2024/0173646 A1
(43) Pub. Date: May 30, 2024

(54) PRODUCT FOR BIOMOLECULE PURIFICATION

(71) Applicant: KANEKA CORPORATION, Osaka-shi, Osaka (JP)

(72) Inventors: Masakatsu NISHIHACHIJO, Takasago-shi (JP); Takuma SUEOKA, Takasago-shi (JP); Hisako YAURA, Takasago-shi (JP)

(73) Assignee: KANEKA CORPORATION, Osaka-shi, Osaka (JP)

(21) Appl. No.: 17/802,192

(22) PCT Filed: Feb. 19, 2021
(86) PCT No.: PCT/JP2021/006359

§ 371 Date: Aug. 25, 2022

(30) Foreign Application Priority Data
Feb. 28, 2020 (JP) 2020-032513

(51) Int. Cl. B01D 15/02; B01D 36/00

(52) U.S. Cl. CPC B01D 15/02; B01D 36/00

(57) ABSTRACT

The objective of the present invention is to provide a container for easily and efficiently purifying a target biomolecule from a cell culture liquid or a body fluid and a method for purifying a target biomolecule by using the container. The purification container for a target biomolecule from a cell culture liquid or a body fluid is characterized by comprising an inlet, a filter and an outlet, and further comprising an adsorbent for adsorbing an impurity and/or an aggregating agent for aggregating an impurity, wherein the filter captures the impurity adsorbed on the adsorbent and/or the impurity aggregated by the aggregating agent.

### REFERENCE TO SEQUENCE LISTING SUBMITTED VIA EFS-WEB

This application includes an electronically submitted sequence listing in .txt format. The .txt file contains a sequence listing entitled “4991-0264PUS1_ST25.txt” created on Sep. 8, 2023 and is 720 bytes in size. The sequence listing contained in this .txt file is part of the specification and is hereby incorporated by reference herein in its entirety.

### TECHNICAL FIELD

The present invention relates to a container for easily and efficiently purifying a target biomolecule from a cell culture liquid or a body fluid and a method for purifying a target biomolecule by using the container.

### BACKGROUND ART

An antibody as a useful biomolecule has been prepared by immunizing a mouse with an antigen, fusing an antibody-producing cell such as splenocyte thereof with a myeloma cell to prepare a hybridoma having replication-competent ability, screening and cultivating only a fusion cell having a target antigenic specificity, and obtaining the antibody from a secretion thereof as a monoclonal antibody. The antibody prepared by this method has a problem that the antibody is perceived as an antigen in a human body. Antibodies have therefore been prepared mainly by a transformation method since a transformation method using a plasmid having a gene encoding a target antibody has been put to practical use. Not only a human antibody can be directly prepared but also an antibody-like molecule composed of only a part necessary for antigen recognition, cytotoxic activity or the like among antibodies can be prepared by a transformation method.

A target virus is transmitted to a cell and increased for basic research and preparation of vaccines, since there are few pharmaceutical agents effective against a virus. A virus and a plasmid are useful as a vector. A peptide such as insulin is known as a hormone, which is an important messenger in a living body, and such a peptide may be prepared by a transformation method in some cases.

A cell is generally shaken for cultivation in a liquid culture medium in a flask. Since a cell may be broken during shaking cultivation due to effects of waves, Patent documents 1 to 3 disclose a mechanism for stirring the inside of a culture device with sealing the device. Patent documents 1 to 3 however do not disclose how to separate cells and culture medium.

Some important biomolecules may be produced by a transformation method as described above, but transformation methods have problems because the methods create troubles with purification of a target biomolecule from a cultivated cell or a culture medium. Specifically, a target biomolecule has to be purified by cultivating a transformed cell, breaking the cell or dissolving the cell with a surfactant, removing the cell fragment with filtration, and subjecting the filtrate containing the target biomolecule to chromatography, such as affinity chromatography, ion-exchange chromatography and gel filtration chromatography.

Patent document 4 discloses a cell separation device having an inlet for a liquid containing a cell, a liquid outlet, a flow path to deposit the cell and a cell outlet at the lower part of the depositing flow path.

### PRIOR ART DOCUMENT

### Patent Document

Patent document 1: WO 2005/104706
Patent document 2: WO 2006/116139
Patent document 3: WO 2013/040161
Patent document 4: Japanese Unexamined Utility Model Application Publication No. H2-131898

### DISCLOSURE OF THE INVENTION

### Problems to be Solved by the Invention

A device for cultivating a cell producing a useful biomolecule and a device for separating a cell and a culture liquid are known as described above.

If a part of an impurity can be removed after a cell or a cell fragment is removed from a culture liquid or the like containing a useful biomolecule and before the useful biomolecule is purified by chromatography, a load on chromatography can be reduced and the useful biomolecule can be purified more efficiently.

The objective of the present invention is to provide a container for easily and efficiently purifying a target biomolecule from a cell culture liquid or a body fluid and a method for purifying a target biomolecule by using the container.

### Means for Solving the Problems

The inventors of the present invention made extensive studies to solve the above-described problem. As a result, the inventors completed the present invention by finding that a cell culture liquid can be separated from a cell, a cell fragment or the like and a part of an impurity in the culture liquid can be easily and efficiently removed by particularly supplying the culture liquid with an adsorbent and/or an aggregating agent into a container having an outlet equipped with a filter and then discharging the culture liquid containing a target biomolecule.

Hereinafter, the present invention is described.
[1] A purification container for a target biomolecule from a cell culture liquid or a body fluid,
   comprising an inlet, a filter and an outlet,
   comprising an adsorbent for adsorbing an impurity and/or an aggregating agent for aggregating an impurity,
   wherein the filter captures the impurity adsorbed on the adsorbent and/or the impurity aggregated by the aggregating agent.
[2] The purification container according to the above [1], wherein the adsorbent and/or the aggregating agent is one or more selected from a water-insoluble inorganic compound, an ion-exchange resin, a hydrophobic interaction resin, a hydrophilic interaction resin, an affinity resin and activated carbon.
[3] The purification container according to the above [2], wherein the water-insoluble inorganic compound is one or more selected from magnesium carbonate, magnesium hydroxide, magnesium oxide, calcium sulfate and aluminum oxide.
[4] The purification container according to any one of the above [1] to [3], wherein the impurity is one or more selected from a cell, a cell fragment, a cell lysate, a nucleic acid and a nucleic acid-binding protein.
[5] The purification container according to any one of the above [1] to [4], wherein the target biomolecule is one or more selected from a virus, a virus-like particle, a vaccine, an antibody, an antibody-like molecule, an antibody-binding protein, a hormone, a cytokine, a growth factor, an enzyme and a plasma protein.
[6] The purification container according to any one of the above [1] to [5], wherein a pore diameter of the filter is 0.1 μm or more and 100 μm or less.
[7] The purification container according to any one of the above [1] to [6], further comprising a means for stirring the cell culture liquid or the body fluid and the adsorbent and/or the aggregating agent.
[8] A method for purifying the target biomolecule from the cell culture liquid or the body fluid, the method comprising the steps of:
   supplying the cell culture liquid or the body fluid into the inlet of the purification container according to any one of the above [1] to [7], and
   contacting the cell culture liquid or the body fluid with the adsorbent and/or the aggregating agent.
[9] A method for purifying a target biomolecule from a cell culture liquid or a body fluid, the method comprising the step of supplying the cell culture liquid or the body fluid into an inlet of a purification container comprising a filter and an outlet and an adsorbent for adsorbing an impurity and/or an aggregating agent for aggregating an impurity to contact the cell culture liquid or the body fluid with the adsorbent and/or the aggregating agent.
[10] The method according to the above [9], wherein the cell culture liquid or the body fluid is supplied into the inlet with closing the outlet.
[11] The method according to the above [9] or [10], wherein the purification container further comprises a stirring means to accelerate contact of the cell culture liquid or the body fluid with the adsorbent and/or the aggregating agent.
[12] The method according to the above [10], the method further comprising the step of opening the outlet to discharge the cell culture liquid or the body fluid from the purification container and capture the adsorbent and/or the aggregating agent by the filter.
[13] The method according to any one of the above [9] to [12], wherein the adsorbent and/or the aggregating agent is one or more selected from a water-insoluble inorganic compound, an ion-exchange resin, a hydrophobic interaction resin, a hydrophilic interaction resin, an affinity resin and activated carbon.
[14] The method according to the above [13], wherein the water-insoluble inorganic compound is one or more selected from magnesium carbonate, magnesium hydroxide, magnesium oxide, calcium sulfate and aluminum oxide.
[15] The method according to any one of the above [9] to [14], wherein the impurity is one or more selected from a cell, a cell fragment, a cell lysate, a nucleic acid and a nucleic acid-binding protein.

[16] The method according to any one of the above [9] to [15], wherein the target biomolecule is one or more selected from a virus, a virus-like particle, a vaccine, an antibody, an antibody-like molecule, an antibody binding protein, a hormone, a cytokine, a growth factor, an enzyme and a plasma protein.
[17] The method according to any one of the above [9] to [16], wherein a pore diameter of the filter is 0.1 μm or more and 100 μm or less.
[18] Use of a container for purifying a target biomolecule from a cell culture liquid or a body fluid,
   wherein the container comprises an inlet, a filter and an outlet,
   wherein the container comprises an adsorbent for adsorbing an impurity and/or an aggregating agent for aggregating an impurity, and
   wherein the filter captures the impurity adsorbed on the adsorbent and/or the impurity aggregated by the aggregating agent.
[19] The use according to the above [18], wherein the adsorbent and/or the aggregating agent is one or more selected from a water-insoluble inorganic compound, an ion-exchange resin, a hydrophobic interaction resin, a hydrophilic interaction resin, an affinity resin and activated carbon.
[20] The use according to the above [19], wherein the water-insoluble inorganic compound is one or more selected from magnesium carbonate, magnesium hydroxide, magnesium oxide, calcium sulfate and aluminum oxide.
[21] The use according to any one of the above [18] to [20], wherein the impurity is one or more selected from a cell, a cell fragment, a cell lysate, a nucleic acid and a nucleic acid-binding protein.
[22] The use according to any one of the above [18] to [21], wherein the target biomolecule is one or more selected from a virus, a virus-like particle, a vaccine, an antibody, an antibody-like molecule, an antibody binding protein, a hormone, a cytokine, a growth factor, an enzyme and a plasma protein.
[23] The use according to any one of the above [18] to [22], wherein a pore diameter of the filter is 0.1 μm or more and 100 μm or less.
[24] The use according to any one of the above [18] to [23], further comprising a means for stirring the cell culture liquid or the body fluid and the adsorbent and/or the aggregating agent.

### Effect of the Invention

For example, a culture liquid containing a target biomolecule can be separated very easily from a cell and an impurity by supplying the culture liquid containing the cell producing the target biomolecule or the broken cell into the purification container for a biomolecule according to the present invention and then discharging the culture liquid, since the impurity contained in the culture liquid is selectively adsorbed on the adsorbent or aggregated by the aggregating agent. The present invention is therefore industrially very useful, since a target biomolecule produced by a transformation method or the like can be efficiently purified from a cell culture liquid or the like containing the target biomolecule by the present invention.

### BRIEF DESCRIPTION OF THE DRAWINGS

FIG. 1 is a schematic diagram to demonstrate one embodiment of the container according to the present invention.

FIG. 2 is a schematic diagram to demonstrate one embodiment of the container according to the present invention.

FIG. 3 is a schematic diagram to demonstrate one embodiment of the container according to the present invention.

FIG. 4 is a schematic diagram to demonstrate one embodiment of the container according to the present invention.

FIG. 5 is a graph to demonstrate the measurement result of particle size distribution of commercially available basic magnesium carbonate.

FIG. 6 is a graph to demonstrate the adeno-associated virus recovery rate, the impurity protein removal rate and the impurity DNA removal rate in the case where an adeno-associated virus culture liquid is treated in various concentrations of a nucleolytic enzyme and various additive amounts of basic magnesium carbonate for various time.

FIG. 7 is a graph to demonstrate the impurity protein removal rate in the case where an adeno-associated virus culture liquid is treated with basic magnesium carbonate for various time.

### MODE FOR CARRYING OUT THE INVENTION

The purification container of the present invention is used for purifying a target biomolecule from a cell culture liquid or a body fluid. The cell culture liquid or the body fluid to be supplied into the present invention container may be hereinafter referred to as “liquid to be treated” in some cases.

The material of the purification container according to the present invention is not particularly restricted as long as the supplied liquid to be treated is not leaked, and is exemplified by a resin such as polyethylene, polypropylene, polyacrylate, polycarbonate, polystyrene, polyamide and polyester; a metal such as stainless and aluminium; an inorganic material such as glass and ceramic; and a composite material such as a glass fiber impregnated with a resin and a carbon fiber impregnated with a resin. One of the materials may be used alone, and a multi-stratified material may be used in order to prevent non-specific adsorption of a target biomolecule on the container and prevent elution and leakage from the material.

When the container is flexible, the volume of the container before the liquid to be treated is supplied can be reduced and the container can be easily transported. In addition, after the liquid to be treated is supplied into the flexible container, the liquid to be treated can be contacted with the adsorbent and/or the aggregating agent by transforming the container even without stirring. On the one hand, the non-flexible container composed of, for example, a metal, is suitable as a large container for the mass production of a target biomolecule.

The shape of the container may be appropriately selected, and preferably has a bottom and is self-standing, such as cylindrical shape, cone shape and cuboid. Even when the container is flexible, the container having a bottom can be self-standing by supplying the liquid to be treated thereto. In particular, the relatively small flexible container may have a so-called bag-like shape of which each side is sealed. When the container is flexible, an outer envelope container and a support rack may be used in order to, for example, maintain the shape of the container and stand the container for supplying a liquid to be treated and maintain the shape of the container by supporting the weight and pressure in the condition that the inside of the container is filled with a liquid to be treated. An example of the material of such a support rack and an outer envelope container includes a metal such a stainless; and a resin such as polyethylene and polypropylene. The outer envelope container and the support rack may be equipped with a tube for circulating a heating medium and a cooling medium to adjust the temperature of a liquid to be treated inside of the container.

The container of the present invention preferably contains an adsorbent and/or an aggregating agent and the inside of the container is preferably sterile before use, since the container can be used for producing an active ingredient of a biopharmaceutical product. The container itself, the adsorbent and/or the aggregating agent, or the like may be sterilized by any methods suitable for each in order to create a sterile environment inside of the container. An example of a sterilization method includes methods using heating, chemical treatment, gamma rays, ultraviolet rays and ethylene oxide gas. A material of the container may be selected depending on a sterilization method.

The volume of the container may be adequately adjusted and may be adjusted to, for example, 10 mL or more and 5000 L or less. The volume of 10 mL or more and 10 L or less is suitable for crude purification of a target biomolecule in a laboratory. The volume of 10 L or more is applicable to mass production of a target biomolecule. When the volume is 5000 L or less, the liquid to be treated can be successfully stirred in the container even in the case where an amount of the liquid to be treated is large in mass production. The volume of the bag-shaped container corresponds to the maximum volume of water that can be inserted therein.

The purification container of the present invention comprises an inlet to supply the liquid to be treated into the container. The inlet is not particularly restricted as long as the liquid to be treated can be supplied into the container through the inlet. The size of the inlet may be adequately adjusted depending on an implementation scale or the like. For example, the diameter thereof may be 1 mm or more and 5 cm or less.

The inlet may be a simple pore formed in the upper part of the container. An element to easily supply the liquid to be treated may be attached to the inlet, and a tube may be attached to the inlet to supply the liquid to be treated from, for example, a tank or a fermenter to temporarily store the liquid to be treated. The material of such an element and a tube may be the same as or different from the material of the container. In addition, the inlet may be capped or the tube led to the inlet is provided with a valve in order to inhibit the leakage of the liquid after the liquid to be treated is supplied into the container.

The inlet may be equipped with a prefilter through which a target biomolecule can pass but which can prevent a relatively large impurity such as a cell and a cell fragment from being supplied into the container. The pore diameter of such a prefilter may be adjusted to, for example, 100 μm or more and 5 mm or less.

The container of the present invention comprises an outlet in the lower part to discharge the liquid treated by the adsorbent and/or the aggregating agent. The outlet is not particularly restricted as long as the treated liquid can be discharged from the container. The size of the outlet may be adequately adjusted depending on an implementation scale or the like. For example, the diameter thereof may be 1 mm or more and 5 cm or less. The term “treatment” means that the liquid to be treated is supplied into the container of the present invention to be contacted with the adsorbent and/or the aggregating agent and then discharged.

The outlet may be a simple pore formed in the lower part of the container similarly to the inlet. An element to easily discharge the treated liquid may be attached to the outlet, and a tube may be attached to the outlet to transport the treated liquid from the container, for example, to the other device. The material of such an element and a tube may be the same as or different from the material of the container. In addition, the outlet may be capped or the tube led to the outlet is provided with a valve in order to inhibit the leakage of the liquid supplied into the container.

An adsorbent to adsorb an impurity and/or an aggregating agent to aggregate an impurity is provided in the container of the present invention. As a result, an impurity in the cell culture liquid or the body fluid is adsorbed or aggregated and a target biomolecule in the cell culture liquid or the body fluid can be purified by reducing an amount and a concentration of the impurity.

The adsorbent is not particularly restricted as long as the adsorbent does not adsorb or hardly adsorbs a target biomolecule and adsorbs at least a part of an impurity contained in the liquid to be treated. An example of the adsorbent includes an inorganic compound that is insoluble in water, which is a main solvent of the liquid to be treated. Such a water-insoluble inorganic compound does not adsorb or hardly adsorbs an antibody, an antibody-like molecule and a virus and may adsorb DNA and a protein having a relatively large molecular weight, such as a DNA-binding protein. An example of a DNA-binding protein includes histone. The term “insolubility” means solubility degree measured by whether dissolved or not dissolved within 30 minutes when inorganic compound powder is added into purified water and the mixture is vigorously shaken for 30 seconds every 5 minutes at 20±5° C. in this disclosure. Specifically, the term “insolubility” means that an amount of purified water required for dissolving 1 g of the inorganic compound is 100 mL or more. The purified water amount is preferably 1000 mL or more.

An example of the water-insoluble inorganic compound usable as the adsorbent includes a water-insoluble inorganic compound comprising one or more elements selected from magnesium, calcium and aluminum. The water-insoluble inorganic compound is preferably an insoluble carbonate, an insoluble sulfate, insoluble phosphate and oxide containing one or more elements selected from magnesium, calcium and aluminum, is specifically exemplified by one or more selected from magnesium carbonate, magnesium hydroxide, magnesium oxide, calcium sulfate, magnesium phosphate and aluminum oxide, and is more preferably a water-insoluble magnesium salt. For example, basic magnesium carbonate, which is a mixture of magnesium hydroxide and magnesium carbonate, is preferably used. On the one hand, a phosphate salt other than magnesium phosphate, such as calcium phosphate, is not preferred, since the phosphate salt has relatively high water-solubility and may possibly adsorb an antibody, an antibody-like molecule and a virus.

An ion-exchange resin, a hydrophobic interaction resin, a hydrophilic interaction resin and an affinity resin can be also used as the adsorbent usable in the present invention in addition to the water-insoluble inorganic compound. In particular, a resin for a chromatography carrier is preferred, since the resin has a molecular size for easy separation and a surface area for sufficient adsorption of an impurity. An anion-exchange resin is suitable for adsorbing a protein and DNA which are derived from a host cell and negatively charged in a neutral condition due to low isoelectric point. A cation ion-exchange resin is suitable for adsorbing a protein which is derived from a host cell and positively charged in a neutral condition due to high isoelectric point. A hydrophobic interaction resin, a hydrophilic interaction resin and activated carbon are suitable for adsorbing an impurity using the difference of a hydrophobicity degree and a hydrophilicity degree between a target biomolecule and an impurity. When an ion-exchange resin, a hydrophobic interaction resin, a hydrophilic interaction resin or activated carbon is used, a target biomolecule may be possibly also adsorbed on the adsorbent but a condition to inhibit the adsorption of a target biomolecule can be set by adjusting the pH and the salt concentration of the liquid to be treated. A resin that does not adsorb a target biomolecule and adsorbs an impurity can be appropriately selected as an affinity resin. A lysine-immobilized resin can remove ribosome RNA and a heparin-immobilized resin can remove nucleic acid-binding protein respectively. An antibody-immobilized resin prepared by immobilizing an antibody binding to a specific impurity can be also used.

The size of the adsorbent may be appropriately adjusted, and for example, the average particle diameter thereof may be adjusted to 0.1 μm or more and 1000 μm or less. When the average particle diameter is 1000 μm or less, the adsorbent can more efficiently adsorb an impurity due to a sufficiently large specific surface area. When the average particle diameter is 0.1 μm or more, excessive energy for pulverization is not needed. The average particle diameter of 1 μm or more is preferred, since the adsorbent is prevented from being mixed in the liquid treated by the container of the present invention. The average particle diameter is measured by a laser diffraction particle size measuring device in this disclosure, and an average particle diameter is based on a volume, a weight, a number or the like and the average particle diameter is preferably based on a volume.

The aggregating agent is not particularly restricted as long as an impurity contained in the liquid to be treated is deposited by affecting the impurity to decrease the water-solubility thereof or precipitate the impurity by aggregating the impurity. It may be sometimes unclear whether a certain substance is the adsorbent or the aggregating agent, and the difference between the adsorbent and the aggregating agent is not necessarily clear. For example, some substances adsorb an aggregated impurity, and some substances clearly reduce an impurity concentration while it is not clear whether the substances adsorb an impurity or aggregate an impurity. A certain substance can be used as the adsorbent and/or the aggregating agent in the present invention as long as the substance can reduce an impurity concentration in the treated liquid in comparison with the supplied liquid to be treated setting aside the mechanism.

An example of the aggregating agent usable in the present invention preferably includes a water-soluble cation polymer to efficiently aggregate and precipitate DNA and a phospholipid having a negative charge and derived from a cell. A polyamine is particularly preferred as the cation polymer. An example of the polyamine compound includes a diamine compound such as polyallylamine, poly (diallyldimethylammonium chloride), polyethylenimine, polyvinylamine, polylysine, ethylenediamine, 1,2-propanediamine, 1,6-hexamethylendiamine, piperazine, 2,5-dimethylpiperazine, isophoronediamine, 4,4′-dicyclohexylmethanediamine and 1,4-cyclohexanediamine; a polyamine compound such as diethylene triamine, dipropylene triamine and triethylenetetramine; a hydrazine compound such as hydrazine, N,N′-dimethylhydrazine and 1,6-hexamethylene bishydrazine; and a dihydrazide compound such as succinic dihydrazide, adipic dihydrazide, glutaric dihydrazide, sebacic dihydrazide and isophthalic dihydrazide. One of the aggregating agents may be used alone, or two or more aggregating agents may be used in combination. The aggregating agent containing polyallylamine and/or poly (diallyldimethylammonium chloride) is preferred among the above examples, since the aggregating agents are commercially available for a bioprocess and are easily available. When a target biomolecule is involved in an impurity and is aggregated and precipitated, a condition to avoid aggregation and precipitation of a target biomolecule can be set by adjusting the pH and the salt concentration of the liquid to be treated.

The useful amounts of the adsorbent and the aggregating agent may be adjusted depending on an inner capacity or the like of the container and for example, can be adjusted to 5 v/v % or more and 50 v/v % or less to the inner capacity of the container. The adsorbent and/or the aggregating agent can be used in an amount of 0.1 g or more and 20 g or less to 100 mL of the liquid to be treated, and the ratio is preferably 15 g/100 mL or less. In addition, the adsorbent and/or the aggregating agent can be used in an amount of 0.1 mass % or more and 20 mass % or less to the liquid to be treated, and the ratio is preferably 1 mass % or more and 15 mass % or less.

The container of the present invention comprises a filter to capture the adsorbent on which an impurity is adsorbed and/or an aggregate containing an impurity and the aggregating agent, thereby to capture an impurity adsorbed on the adsorbent and/or an impurity aggregated by the aggregating agent, and thus to suppress the leakage of the impurity. The filter also has the function to suppress the leakage of the adsorbent on which an impurity is not adsorbed and/or the solid aggregating agent from the container before the liquid to be treated is supplied into the container.

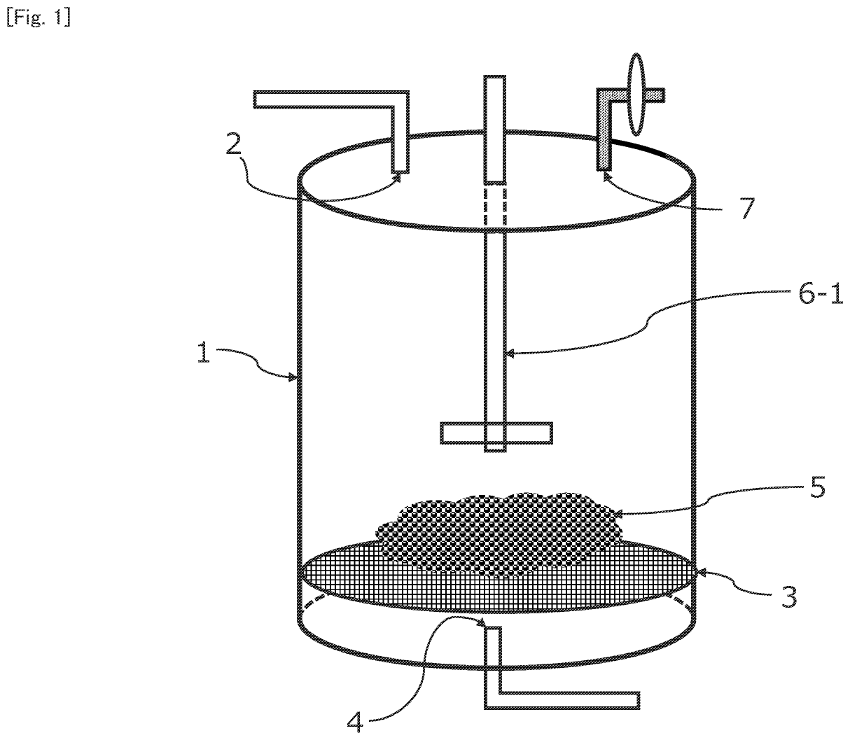

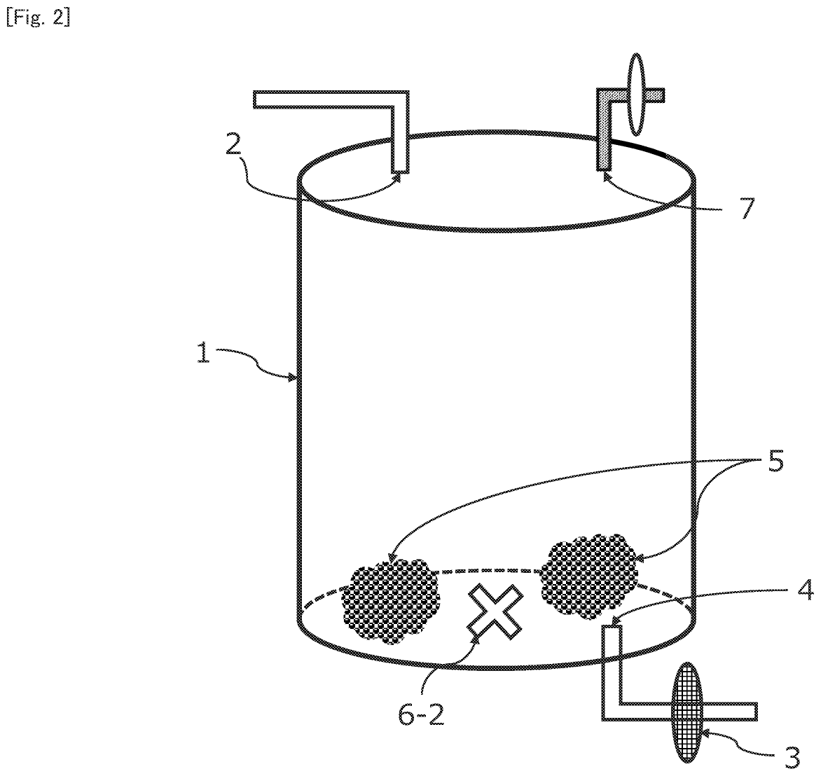

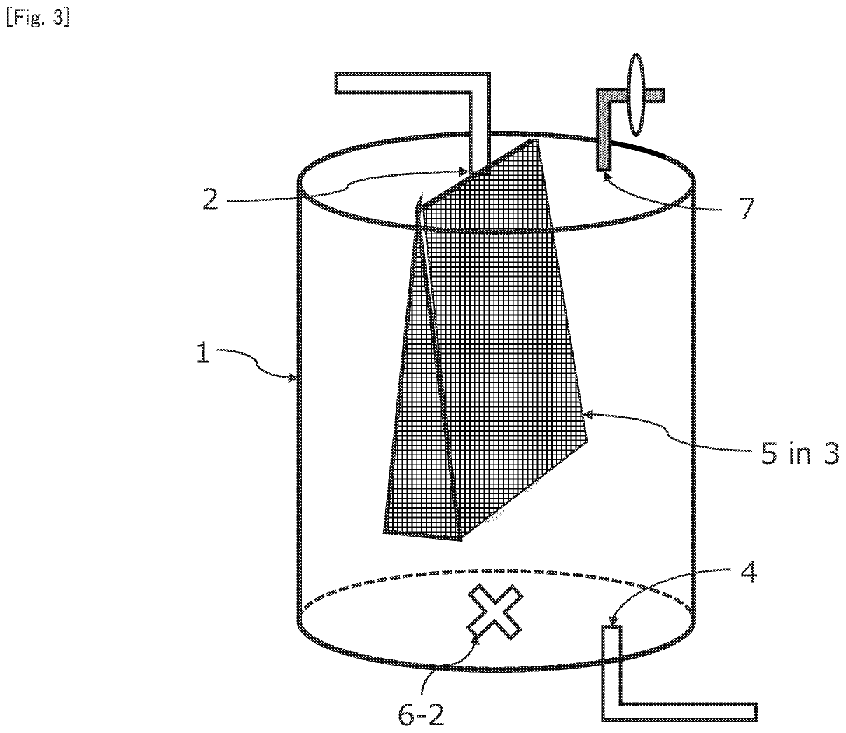

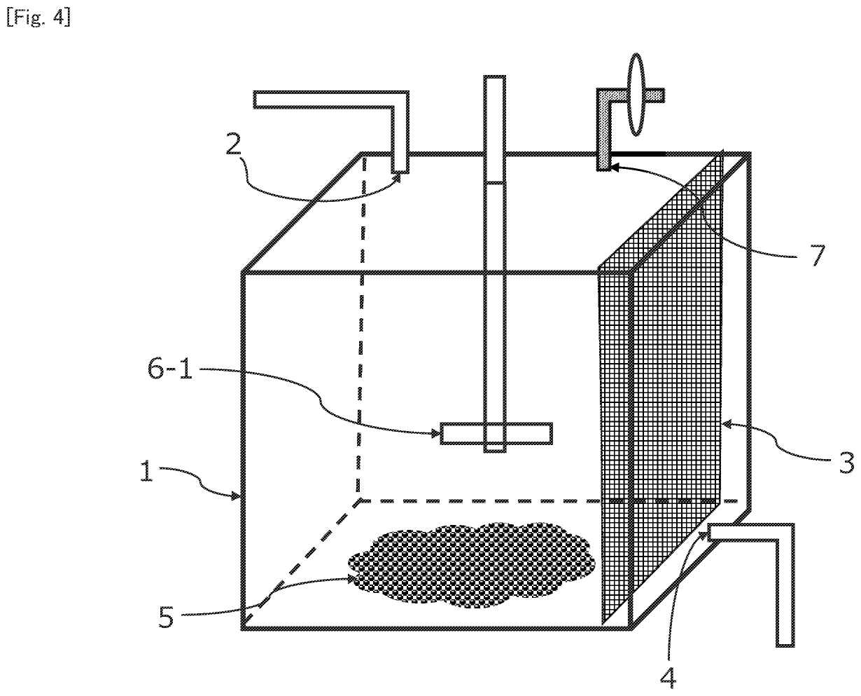

For example, the filter may be placed between the inlet 2 and the outlet 4 in the container 1 as schematically shown in FIG. 1 and FIG. 4. Alternatively, the filter 3 may be set in the tube connecting to the outlet 4 as schematically shown in FIG. 2 to filtrate the treated liquid after the outlet. In addition, the adsorbent and/or the aggregating agent 5 is added into the filter molded into a bag and the filter is added into the container 1 as schematically shown in FIG. 3. A relatively large impurity such as a cell and a cell fragment is preferably removed before the liquid to be treated is supplied into the container by a preliminary filter placed at the inlet or in the tube connecting to the inlet in such a case.

The pore diameter of the filter is preferably adjusted in view of the diameter of an impurity adsorbed on the adsorbent and/or an impurity aggregated by the aggregating agent and the diameter of a target biomolecule so that the impurity adsorbed on the adsorbent and/or the impurity aggregated by the aggregating agent is not permeable and the target biomolecule is permeable. For example, the adsorbent having a smaller pore diameter may have a larger specific surface area and a superior adsorption performance but the adsorbent having a small pore diameter may secondarily become agglutinated in some cases. In addition, it may be difficult to adjust the average particle diameter thereof to be less than 5 μm. Thus, the pore diameter of the filter is preferably adjusted to be 5 μm or less. On the one hand, when the pore diameter of the filter is adjusted to be 0.1 μm or more, a virus and a general protein can sufficiently pass through the filter. The pore diameter is preferably 0.2 μm or more. When the pore diameter of the filter is described in a catalog, the catalog value is adopted as the pore diameter of the filter. When the pore diameter of the filter is not described in a catalog, an average pore diameter estimated on the basis of Gurley permeability may be adopted as the pore diameter of the filter.

The raw material of the filter may be appropriately selected depending on an impurity to be captured, a target biomolecule to be permeable or the like, and is exemplified by a sulfone resin such as polyether sulfone; a polyolefin such as polyethylene and polypropylene; a polyester resin such as nylon; a cellulose such as regenerated cellulose and cellulose acetate; a fluorine resin such as polytetrafluoroethylene and polyvinylidene difluoride; and a ceramic such as alumina and titania.

The container of the present invention preferably comprises a stirring means for stirring a mixture of the liquid to be treated and the adsorbent and/or the aggregating agent in order to adsorb an impurity on the adsorbent and aggregate an impurity by the aggregating agent by accelerating the contact of the liquid to be treated with the adsorbent and/or the aggregating agent after the liquid to be treated is supplied into the container.

An example of the stirring means includes a stirring blade 6-1 that can be moved rotationally or up and down by external force as shown in FIG. 1 and FIG. 4. It is preferred in such a case that the axle comprises one or more stirring blades at the one end and a hub part, the hub part can move rotationally or up and down with the axle, and the container comprises a casing part that is in close contact with the hub part to prevent the leakage of the supplied liquid to be treated and allows the hub part to move rotationally or up and down. The stirring blade may be moved rotationally or up and down manually or mechanically with using a motor or the like.

In addition, an example of the stirring means includes a stirring bar 6-2 and the stirring bar is inserted into the container as shown in FIG. 2 and FIG. 3. The stirring bar can be rotated from the outside using an external device such as a magnetic stirrer.

When the mixture is stirred using a stirring blade, stress may be applied to a target substance due to high shear stress. When the mixture is stirred using a stirring bar, the stirring bar may cause physical damage to a target substance, the container and the adsorbent at the sliding portion. Thus, a stirring means other than a stirring blade and a stirring bar is also preferred. Specifically, a wave may be generated in the liquid to be treated inside of the container by mechanically moving a cradle on which the container is placed up and down like a seesaw, or a stirring flow may be generated in the liquid to be treated inside of the container by putting the container in a shell container and rotating/anti-rotating the shell container in a horizontal direction. In addition, the mixture may be circulated to be mixed by preliminarily connecting two arbitrary parts such as a lower part and an upper part of the container with a pipe and taking the liquid to be treated in and out with a pump such as a tubing pump.

In addition, the mixture of the liquid to be treated and the adsorbent and/or the aggregating agent may be stirred by manually shaking the small container or deforming the flexible container after the liquid to be treated is supplied into the container.

When the outlet is closed to supply the liquid to be treated from the inlet, the liquid to be treated may not be efficiently supplied in some cases since the air inside of the container cannot be discharged. An exhaust vent is preferably provided at the upper part of the container in such a case in order to discharge the air inside of the container when the liquid to be treated is supplied. The exhaust vent is not particularly restricted as long as the air can be discharged through the exhaust vent, and for example, the diameter thereof may be adjusted to 0.1 mm or more and 5 cm or less.

The exhaust vent may be a pore in the upper part of the container, and a member to prevent the leakage of the supplied liquid to be treated, such as a cover, is preferably provided. When the exhaust vent is connected to a tube, a valve may be provided to prevent the leakage of the liquid to be treated.

A target biomolecule contained in the liquid to be treated can be purified by supplying the liquid to be treated into the present invention container to be contacted with the adsorbent and/or the aggregating agent and then discharging the treated liquid. The term “purification” means that the amount of an impurity contained in the liquid treated in the present invention container is reduced in comparison to the amount of an impurity contained in the liquid to be treated before the liquid is treated in the present invention container and the ratio of a relative amount of a target biomolecule to an impurity in the liquid to be treated is increased after the treatment in the present invention container in this disclosure.

Specifically, the liquid to be treated that contains a target biomolecule and an impurity is first supplied from the inlet. The liquid to be treated is preferably supplied from the inlet with closing the outlet of the present invention container at the time. An example of the target biomolecule includes a virus, a virus-like particle, a vaccine, an antibody, an antibody-like molecule, an antibody-binding protein, a hormone, a cytokine, a growth factor, an enzyme and a plasma protein.

The term “virus” means a complex of DNA or RNA and a capsid composed of a protein, wherein the capsid covers the DNA or RNA and the virus does not have a self-replicating ability. An example of a non-enveloped virus includes adeno-associated virus, adenovirus, enterovirus, parvovirus, papovavirus, human papillomavirus, rotavirus, coxsackievirus, sapovirus, norovirus, poliovirus, echovirus, coronavirus, hepatitis A virus, hepatitis E virus, rhinovirus and astrovirus. An adeno-associated virus has an AAV capsid serotype selected from the group consisting of AAV-1, AAV-2, AAV-3, AAV-4, AAV-5, AAV-6, AAV-7, AAV-8, AAV-9, AAV-10, AAV-11, AAV-12, AAV-13, AAV-14, AAV-15 and AAV-16. An example of an enveloped virus includes retrovirus, lentivirus, hemagglutinating virus of Japan, herpes simplex virus, vaccinia virus, measles virus, baculovirus and influenza virus.

The “virus-like particle” means all of or a part of a virus outer shell protein that mainly constitutes a capsid. The virus-like particle does not raise infection concerns, since the virus-like particle does not contain a nucleic acid. But the virus-like particle can be used as an active ingredient of a vaccine, since the virus-like particle causes an immune reaction. In addition, the virus-like particle can be also used for a drug delivery system by introducing a synthesized nucleic acid in the virus-like particle or decomposing and rebuilding the virus-like particle to enclose a physiologically active substance.

The “vaccine” is an inactive form or a part of the structure of a pathogen such as a virus and a bacterium. The vaccine does not raise infection concerns, since the vaccine does not contain a nucleic acid. But the vaccine can be used as an active ingredient, since the vaccine causes an immune reaction. For example, all of or a part of a virus outer shell protein that constitutes a capsid can be used as a virus vaccine.

The term “antibody-like molecule” means an antibody fragment particularly having a function to mediate antigen recognition and an immune reaction. The “antibody or antibody-like molecule” is not particularly restricted and is exemplified by polyclonal antibody, monoclonal antibody, human antibody, humanized antibody, chimeric antibody, single chain antibody, heavy chain antibody, polyvalent antibody, Fab, F(ab′), F(ab′)₂, Fc, Fc-fusion protein, bispecific antibody, heavy chain (H chain), light chain (L chain), single chain Fv (scFv), sc(Fv)₂, disulfide-linked Fv (sdFv), diabody and antibody-like molecule target peptide (micro antibody). The antibody or the antibody-like molecule may be any one of an Fc-containing protein, such as immunoglobulin and Fc-fusion protein having an Fc part, and a low-molecular antibody such as the above-described Fab, F(ab′), F(ab′)₂, Fc, heavy chain (H chain), light chain (L chain), single chain Fv(scFv), Sc(Fv)₂, disulfide-linked Fv(sdFv), single chain antibody, heavy chain antibody, multivalent antibody, bispecific antibody, diabody and antibody-like molecule target peptide (micro antibody) in the present invention.

The antibody-binding protein is not particularly restricted as long as the antibody-binding protein is a protein having a specific binding ability to an antibody and is exemplified by Protein A, Protein G, Protein L, Fcγ receptor and functional variants thereof.

The “hormone”, “cytokine” and “growth factor” are produced in a specific cell and secreted, means a molecule to induce differentiation, motion, secretion, uptake, proliferation or the like even in a small amount by binding to a specific receptor of the other cell with a high affinity, and means a substance particularly composed of a protein in the present invention. An example of the hormone includes insulin, glucagon, somatostatin, growth hormone, parathyroid hormone, prolactin, leptin and calcitonin. An example of the cytokine includes interleukin, interferon (IFN α, IFN β and IFN γ) and tumor necrosis factor (TNF). An example of the growth factor includes epidermal growth factor (EGF), insulin-like growth factor (IGF), transforming growth factor (TGF), nerve growth factor (NGF), brain-derived neurotrophic factor (BDNF), vascular endothelial growth factor (VEGF), granulocyte colony-stimulating factor (G-CSF), granulocyte-macrophage colony-stimulating factor (GM-CSF), platelet-derived growth factor (PDGF), erythropoietin (EPO), thrombopoietin (TPO), fibroblast growth factor (FGF) and hepatocellular growth factor (HGF).

The “enzyme” means a protein that is produced in a cell of a living body and has a catalytic activity specific to chemical change in a living body. The enzyme functions in a very small amount and is a very specific catalyst. In general, the enzyme catalyzes a specific change of a specific group of a substrate. An example of the enzyme includes lipase, protease, steroid synthetase, kinase, phosphatase, xylanase, esterase, methylase, demethylase, oxidase, reductase, cellulase, aromatase, collagenase, transglutaminase, glycosidase and chitinase.

The “plasma protein” means a protein that is dissolved in plasma and is exemplified by thrombin, serum albumin, VII factor, VIII factor, IX factor, X factor and tissue plasminogen activator.

An example of the liquid to be treated by using the container of the present invention includes a culture liquid of a transformed cell producing the above-described target biomolecule, a cell fragment suspension thereof, a cell lysis solution by a surfactant, a culture supernatant obtained by centrifugation or filtration, and extracts therefrom. The cell culture liquid and the treated culture liquid are called together as a cell culture liquid. The liquid to be treated may be also a body fluid containing a target biomolecule. The body fluid means a liquid with which the space between tissues, body cavity, a tube spreading systemically and a circulatory system are filled in an animal body, or a liquid produced by a cell and secreted or excreted inside or out of the body. For example, a body fluid containing a virus may be a chorioallantoic fluid obtained by inoculating the virus strain into the inside of the allantoic cavity of a hen egg to be cultivated and separating.

An example of an impurity contained in the liquid to be treated includes a cell, a cell fragment obtained by disruption and dissolution of a cell, DNA and a DNA-binding protein. A cell and a cell fragment can be removed using the above-described preliminary filter, since a cell and a cell fragment is relatively large. A DNA-binding protein such as histone is relatively larger than the above-described biomolecules, and may be easy to adsorb on the adsorbent or aggregate by the aggregating agent.

Then, the supplied liquid to be treated and the adsorbent and/or the aggregating agent are mixed in the present invention container. The mixture of both may be stirred by the above-described stirring means, shaken by a human in the case of the relatively small container, or the flexible container may be deformed as a mixing means.

A mixing condition may be appropriately adjusted. For example, the temperature for mixing may be an atmospheric temperature and may be specifically adjusted to, for example, 0° ° C. or higher and 40° ° C. or lower. The temperature is preferably 1° C. or higher, more preferably 10° C. or higher or 15° C. or higher, and preferably 30° C. or lower, more preferably 25° C. or lower. The contact time may be adjusted to 1 second or more and 10 hours or less, and is preferably 10 minutes or more and more preferably 30 minutes or more.

After at least a part of an impurity contained in the liquid to be treated is adsorbed on the adsorbent or aggregated by the aggregating agent by mixing the liquid to be treated and the adsorbent and/or the aggregating agent, and then the treated liquid is discharged from the outlet. A relatively large substance such as a cell and a cell fragment, the adsorbent and the aggregate are separated from the treated liquid by the filter at the time. As a result, a target biomolecule is purified, since the amount of a target biomolecule does not change or hardly changes but an impurity is adsorbed on the adsorbent or aggregated by the aggregating agent. A target biomolecule is preferably further purified by subjecting the treated liquid to affinity chromatography, ion-exchange chromatography, gel filtration chromatography or the like, since an impurity is difficult to completely remove by the above-described step.

The present application claims the benefit of the priority date of Japanese patent application No. 2020-32513 filed on Feb. 28, 2020. All of the contents of the Japanese patent application No. 2020-32513 filed on Feb. 28, 2020, are incorporated by reference herein.

### EXAMPLES

Hereinafter, the present invention is described in more detail with Examples. The present invention is however not restricted to the following Examples in any way, and it is possible to work the present invention according to the Examples with an additional appropriate change within the range of the above descriptions and the following descriptions. Such a changed embodiment is also included in the technical scope of the present invention. Commercially available reagents were used in the following Examples unless otherwise stated.

### Example 1: Rough Purification of Virus

### (1) Preparation of Adeno-Associated Virus (AAV)-Producing Cell

A plasmid which produced AAV2 and which expressed VENUS (GenBank: ACQ43955.1), which is a variant of fluorescent protein GFP, was prepared using a kit to prepare AAV vector (“AAVpro® Helper Free System” manufactured by Takara Bio).

A plasmid was prepared using a transfection reagent (“Polyethylenimine MAX” manufactured by Polysciences, MW: 40,000) . The plasmid was transfected into a cultivated HEK293 cell to produce AAV. The cell was exfoliated after the cultivation to obtain the cell culture liquid.

### (2) Preparation of Nucleic Acid Degradative Treatment Liquid

A surfactant (“Triton® X-100”) was added to the AAV culture liquid obtained in the above-described (1) in a concentration of 0.1 v/v %, and the mixture was stirred in ice for 20 minutes to lyse the cell. To the thus obtained cell lysate liquid, 7.5 v/v % 1 M magnesium chloride aqueous solution and 0.1 v/v % endonuclease manufactured by KANEKA were added. The mixture was left to stand at 37° C. for 30 minutes to degrade the nucleic acid derived from the cell. The mixture was regarded as nucleolytic degradation-treated liquid, and the amounts of AAV and the total protein in the liquid were measured. The result is shown in Table 1. The concentration of AAV was determined using an AAV titer measuring kit (“AAVpro® Titration Kit (for Real Time PCR) Ver. 2” manufactured by Takara Bio) , and the total protein concentration was determined using bovine serum albumin (BSA) as a standard product and a protein colorimetric assay reagent (“Pierce 660 nm Protein Assay Reagent” manufactured by Thermo Fisher Scientific).

### (3) Removal of Impurity by Water-Insoluble Magnesium Compound

Light basic magnesium carbonate (manufactured by Wako Pure Chemical, 59 g) and a stirring bar were added into a cylindrical polyethylene container having an outside diameter of 12.7 cm×an overall height of 23.5 cm. The nucleolytic degradation-treated liquid obtained in the above-described (2) (590 g) was further added thereto, and the mixture was stirred at room temperature for 1 hour. The ratio of the light basic magnesium carbonate to the nucleolytic degradation-treated liquid was 10 w/w %.

Then, the above mixture liquid was filtrated through a polyether sulfone filter (“Nalgene Rapid-Flow Sterile Disposable Filter Units” manufactured by Thermo Scientific, pore diameter: 0.2 μm), and the obtained filtrate was regarded as a first filtrate. Dulbecco's phosphate buffered saline (manufactured by Sigma-Aldrich, 59 mL, hereinafter abbreviated as “PBS”) was added to the remaining filtration residue and was filtrated, and the obtained filtrate was regarded as a second filtrate. The first filtrate and the second filtrate were mixed, and the mixture was regarded as a pre-treated liquid 1, and the amounts of AAV and the total protein were measured similarly to the above. Also, the amounts of AAV and the total protein in the nucleolytic degradation-treated liquid were similarly measured. The result is shown in Table 1.

TABLE 1

| | Liquid | Total protein | AAV | |
|---|---|---|---|---|
| | weight | concentration | concentration | AAV amount |
| Nucleolytic | 590 g | 0.37 mg/mL | 1.73 × 10¹⁰ vg/mL | 1.02 × 10¹³ vg |
| degradation-treated liquid | | | | |
| Pre-treated liquid 1 | 605 g | 0.02 mg/mL | 1.98 × 10¹⁰ vg/mL | 1.20 × 10¹³ vg |

It could be confirmed from the result shown in table 1 that an AAV amount was not decreased by the treatment. On the one hand, it could be confirmed that an impurity protein derived from a cell can be easily removed by the present invention method, since a total protein amount is remarkably reduced.

It was also confirmed that not only a cell lysate but also a basic magnesium carbonate particle on which an impurity protein may be adsorbed can be separated from a target AAV by using a filter of 0.2 μm.

### Comparative Example 1: Rough Purification of Virus Using Depth Filter and Ultrafiltration Membrane

A depth filter (“Supracap 50 capsule with V100P” manufactured by PALL, effective filtration area: 22 cm², pore diameter: 1 to 3 μm) was rinsed with 20 mM Tris+120 mM NaCl aqueous solution (pH 8.0). Then, the nucleolytic degradation-treated liquid (592 g) obtained in Example 1 (2) was filtrated using the depth filter, and the obtained filtrate was recovered as a first filtrate. Further, 20 mM Tris+120 mM NaCl aqueous solution (pH 8.0, 50 mL) was supplied to the depth filter, and the obtained filtrate was obtained as a second filtrate. The obtained first filtrate and the second filtrate were mixed as a clarified liquid.

The clarified liquid (647 mL) was concentrated to about 50 mL using a pump system (“AKTA flux S” manufactured by GE Healthcare) and an ultrafiltration membrane (“Suspended-Screen Ultrafiltration Cassettes with Omegatm Membrane: Centramate” manufactured by PALL, membrane area: 0.02 m², nominal molecular weight cut off: 300 K). The buffer was exchanged by continuously adding about eight times amount of 20 mM Tris+120 mM NaCl+0.005% Tween 20+1 mM MgCl₂ aqueous solution (pH 8.1) as a dialysate to the concentrated liquid with maintaining the amount of the concentrated liquid. The dialyzed liquid in the system was obtained after the dialysis. In addition, the system was washed with the dialysate, and the washing liquid was obtained.

The dialyzed liquid and the washing liquid were mixed, and the mixture was filtrated using a polyether sulfone filter (“Nalgene Rapid-Flow Sterile Disposable Filter Units” manufactured by Thermo Scientific, pore diameter: 0.2 μm) to obtain a pre-treated liquid 2. The amounts of AAV and the total protein in the pre-treated liquid 2 were determined similarly to Example 1(1). The result is shown in Table 2.

TABLE 2

| | Liquid | Total protein | | AAV | AAV |
|---|---|---|---|---|---|
| | amount | concentration | | concentration | amount |
| Nucleolytic | 592 g | 0.37 | mg/mL | 1.73 × 10¹⁰ vg/mL | 1.02 × 10¹³ vg |
| degradation-treated liquid | | | | | |
| Clarified liquid | 646 g | 0.29 | mg/mL | 1.77 × 10¹⁰ vg/mL | 1.15 × 10¹³ vg |
| Concentrated liquid | 577 g | 0.22 | mg/mL | 8.57 × 10⁹ vg/mL | 4.94 × 10¹² vg |
| Dialyzed liquid | 422 g | 0.03 | mg/mL | 8.38 × 10⁹ vg/mL | 3.54 × 10¹² vg |
| Pre-treated liquid 2 | 102 g | <0.01 | mg/mL | 2.49 × 10¹⁰ vg/mL | 2.55 × 10¹² vg |

The AAV amount was not changed, the total protein concentration was not remarkably reduced and most part of an impurity protein remained by the filtration using a depth filter as the result shown in Table 2.

The total protein concentration was remarkably reduced, the impurity protein could be also removed, and the AAV amount was also reduced by the treatment using an ultrafiltration membrane. Since AAV was observed in the filtrate, the AAV permeated the ultrafiltration membrane due to large pores.

It was found from the above result that rough purification using a depth filter and an ultrafiltration membrane required multistep treatment and a complicated operation, and is at risk for low AAV recovery rate, though a total protein amount can be reduced.

### Comparative Example 2: Purification of Virus by Affinity Chromatography

The nucleolytic degradation-treated liquid obtained in Example 1 (2) was centrifuged, and the supernatant was filtrated using a polyether sulfone filter (“Nalgene Rapid-Flow Sterile Disposable Filter Units” manufactured by Thermo Scientific, pore diameter: 0.2 μm). The filtrate was regarded as a clarified liquid without pretreatment. An amount of a liquid containing an AAV amount of 1×10¹² vg was taken from the clarified liquid without pretreatment, and a nine times amount of an equilibration buffer was mixed therewith. The mixture was filtrated using a filter.

The obtained filtrate was subjected to affinity chromatography in the following condition to purify AAV. An amount of AAV in the eluate was determined by quantitative PCR, and a recovery rate to the supplied AAV amount was calculated. In addition, an amount of a liquid containing an AAV amount of 1×10¹² vg was taken from the pre-treated liquid 1 of Example 1 and the pre-treated liquid 2 of Comparative example 1, and a nine times amount of an equilibration buffer was mixed therewith for comparison. The mixture was filtrated using a filter. The obtained filtrate was also subjected to the same affinity chromatography to determine an AAV amount and calculate an AAV recovery rate. The result is shown in Table 3.

### <Chromatography Condition>

Column: Tricron 5/50 manufactured by GE Healthcare
Carrier: POROS CaptureSelect AAVX manufactured by Thermo Scientific, 1 mL
Flow rate: 0.5 mL/min
Equilibration buffer: 20 mM tris (hydroxymethyl) aminomethane-hydrochloride buffer, 0.5 M sodium chloride (pH 8.0)
Elution buffer: 0.1 M citrate buffer (pH 2.1)

TABLE 3

| | | AAV amount | AAV amount | |
|---|---|---|---|---|
| | | before | after | Recovery |
| Supplied liquid | | treatment | treatment | rate |
| Pre-treataed | Example 1 | 8.0 × 10¹¹ vg | 7.2 × 10¹¹ vg | 90% |
| liquid 1 | | | | |
| Pre-treataed | Comparative | 8.6 × 10¹¹ vg | 6.1 × 10¹¹ vg | 71% |
| liquid 2 | example 1 | | | |
| Without | Comparative | 1.1 × 10¹² vg | 6.0 × 10¹¹ vg | 57% |
| pretreatment | example 2 | | | |

It was found from the result shown in Table 3 that a recovery rate in a latter affinity chromatography purification from the clarified liquid obtained by the pretreatment of the present invention is higher in comparison with a pre-treated liquid obtained by a depth filter and an ultrafiltration membrane (Comparative example 1) and a clarified liquid without pretreatment (Comparative example 2).

### Example 2: Particle Size Distribution Measurement of Material to Remove Impurity

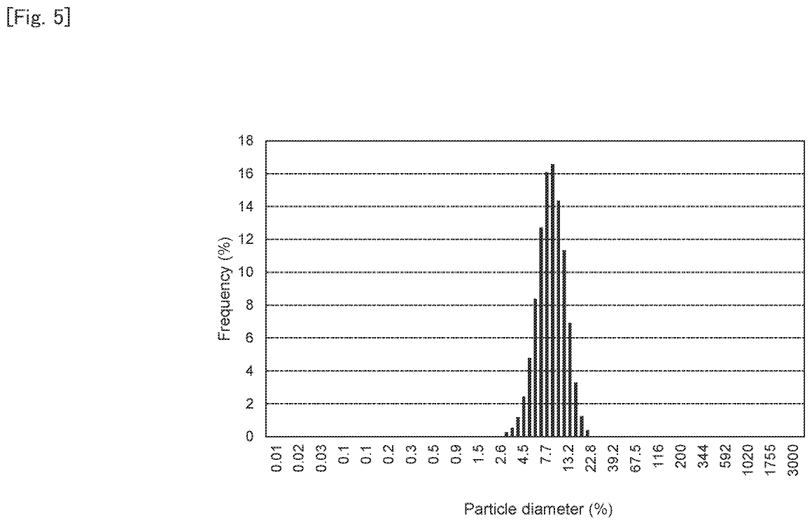

Light basic magnesium carbonate used in Example 1 (3) was dispersed in water, and the particle size distribution was measured by a wet method using a particle size distribution analyzer (“Partica LA-960” manufactured by HORIBA). The result is shown in FIG. 5.

As a result, the median diameter was 7.9 μm, and the volumetric basis 10% diameter was 5.2 μm. Thus, 90% or more of the above basic magnesium carbonate may be separated by using a filter having a pore diameter of 5 μm or less. Since AAV permeated a filter having a pore diameter of 0.2 μm in Example 1, the basic magnesium carbonate particle and AAV can be separated by using a filter of 0.2 to 5 μm.

### Example 3: Evaluation of Treatment Condition Using Water-Insoluble Magnesium Compound

An AAV culture liquid was treated by the treatment condition using a water-insoluble magnesium compound, specifically an additive amount, a treatment time and a concentration of a nucleolytic enzyme demonstrated in Table 4, to evaluate an AAV recovery rate, a protein removal rate and a DNA removal rate.

A surfactant (“Triton® X-100”) was added to the AAV culture liquid obtained in Example 1(1) in a concentration of 0.1 v/v %, and the mixture was stirred in ice for 20 minutes to lyse the cell. To the thus obtained cell lysate liquid, 7.5 v/v % 1 M magnesium chloride aqueous solution and endonuclease manufactured by KANEKA in a final concentration of 50 U/mL, 5 U/mL or 0.5 U/mL were added. The mixture was left to stand at 37° C. for 30 minutes to degrade the nucleic acid derived from a cell as a nucleolytic degradation-treated liquid. Light basic magnesium carbonate manufactured by Wako Pure Chemical was added to 20 mL of each nucleolytic degradation-treated liquid in a concentration of 10 w/v %, 5 w/v % or 1 w/v %, and the mixture was shaken to be stirred at room temperature for 1 minute, 10 minutes or 60 minutes. The treated liquid was filtrated using a polyether sulfone filter (“Nalgene Rapid-Flow Sterile Disposable Filter Units” manufactured by Thermo Scientific, pore diameter: 0.2 μm). The thus obtained filtrate is called as a first filtrate. Dulbecco's phosphate buffered saline (manufactured by Sigma-Aldrich, 2 mL, hereinafter abbreviated as “PBS”) was added to the remaining filtration residue and was filtrated, and the obtained filtrate was hereinafter called as a second filtrate. The first filtrate and the second filtrate were mixed, and the mixture was called as a pre-treated liquid 3. An AAV amount and a total protein amount in the pre-treated liquid 3 were determined similar to Example 1(2) .

In addition, an amount of the remaining DNA derived from HEK293 cell in the pre-treated liquid 3 was determined in reference to the description of J. Phrama. Biomed. Anal. (2014), 100, 145-149. Specifically, the amount was analyzed using primer 1: GAGGCGGGCGGATCA (SEQ ID No: 1), primer 2: CCCGGCTAATTTTTGTATTTTTAGTAG (SEQ ID No: 2), a reagent set for real-time PCR (“Power SYBRTM Green PCR Master Mix” manufactured by Life Technologies) and a real-time PCR system (“QuantStudio 3” manufactured by Life Technologies). A calibration curve was prepared using Human Genomic DNA manufactured by GenScript as a standard product of DNA derived from a human to determine an amount of DNA.

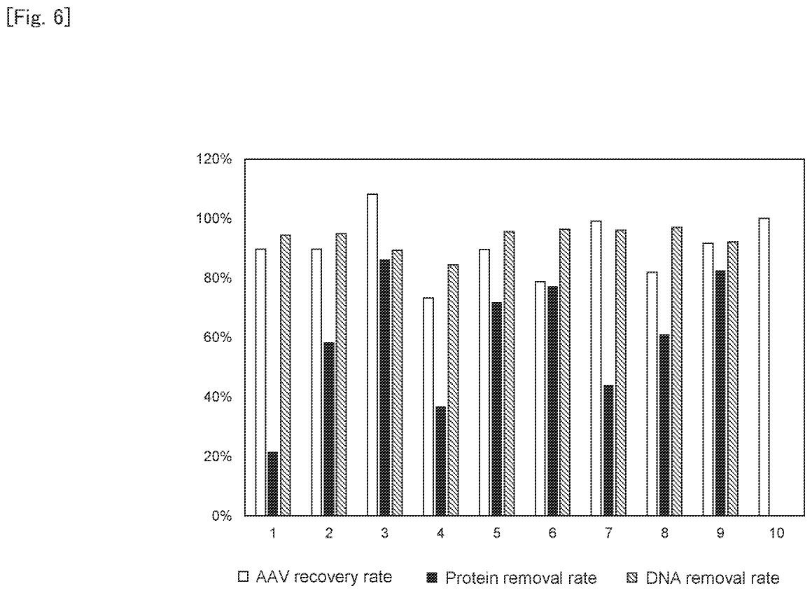

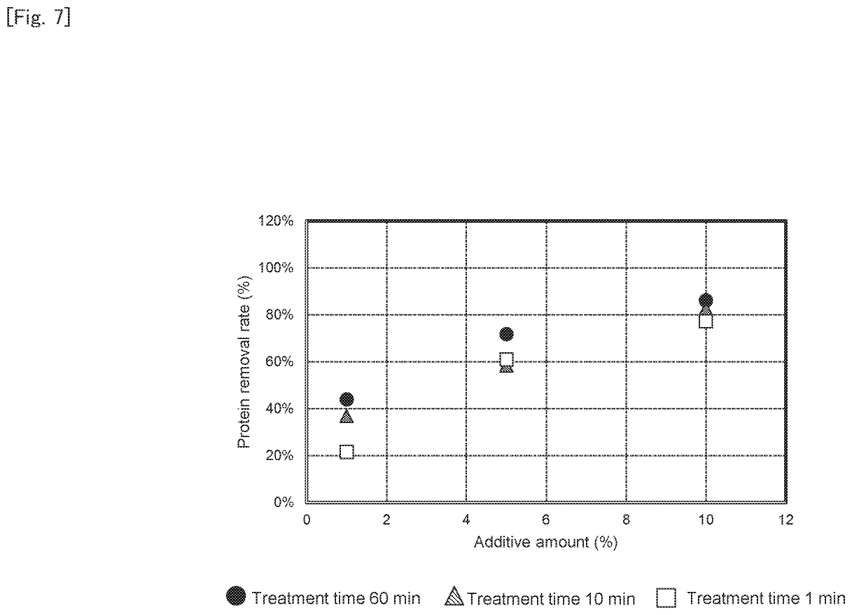

Furthermore, an AAV amount, a protein amount and a DNA amount were similarly determined except that the nucleolytic degradation-treated liquid treated with 50 U/mL endonuclease was not treated with basic magnesium carbonate as a control. In addition, an AAV recovery rate of each treated liquid in the case where the AAV amount of the control was regarded as 100% was calculated. A protein removal rate and a DNA recovery rate in the case where each of the protein recovery rate and the DNA recovery rate of the control were regarded as 0% were calculated. The result is shown in Table 4, FIG. 6 and FIG. 7.

TABLE 4

| | Nucleo- | | | | | | | |
|---|---|---|---|---|---|---|---|---|
| | lytic | | Basic magnesium | | | AAV | | |
| | enzyme | | carbonate | | | recov- | Protein | DNA |
| Test | concen- | | Additive | Treatment | | ery | removal | removal |
| No. | tration | | amount | time | | rate | rate | rate |
| 1 | 0.5 | U/mL | 1% | 1 | min | 90% | 22% | 94% |
| 2 | 0.5 | U/mL | 5% | 10 | min | 90% | 58% | 95% |
| 3 | 0.5 | U/mL | 10% | 60 | min | 108% | 86% | 89% |
| 4 | 5 | U/mL | 1% | 10 | min | 73% | 37% | 84% |
| 5 | 5 | U/mL | 5% | 60 | min | 90% | 72% | 96% |
| 6 | 5 | U/mL | 10% | 1 | min | 79% | 77% | 96% |
| 7 | 50 | U/mL | 1% | 60 | min | 99% | 44% | 96% |
| 8 | 50 | U/mL | 5% | 1 | min | 82% | 61% | 97% |
| 9 | 50 | U/mL | 10% | 10 | min | 92% | 83% | 92% |

| | | | | | | | |
|---|---|---|---|---|---|---|---|
| Control | 50 | U/mL | — | — | 100% | 0% | 0% |

It was found that DNA can be efficiently removed as the DNA recovery rate was 84% or more in any conditions, even when the additive amount, the treatment time and the nucleolytic enzyme concentration were at the lowest level in each condition. In addition, a protein recovery rate tends to be higher in the case where an additive amount of basic magnesium carbonate is larger with respect to a protein concentration as FIG. 7. Thus, a larger additive amount is preferred in order to efficiently remove a protein.

### REFERENCE SIGNS LIST

1: Container
2: Inlet
3: Filter
4: Outlet
5: Adsorbent and/or Aggregating agent
6-1: Stirring blade, 6-2: Stirring bar
7: Exhaust outlet

1. A purification container for a target biomolecule from a cell culture liquid or a body fluid, said container comprising:
an inlet, a filter and an outlet,
an adsorbent for adsorbing an impurity and/or an aggregating agent for aggregating an impurity,
wherein the filter captures the impurity adsorbed on the adsorbent and/or the impurity aggregated by the aggregating agent.

2. The purification container according to claim 1, wherein the adsorbent and/or the aggregating agent is at least one member selected from the group consisting of a water-insoluble inorganic compound, an ion-exchange resin, a hydrophobic interaction resin, a hydrophilic interaction resin, an affinity resin and activated carbon.

3. The purification container according to claim 2, wherein the water-insoluble inorganic compound is at least one member selected from the group consisting of magnesium carbonate, magnesium hydroxide, magnesium oxide, calcium sulfate and aluminum oxide.

4. The purification container according to claim 1, wherein the impurity is at least one member selected from the group consisting of a cell, a cell fragment, a cell lysate, nucleic acid and a nucleic acid-binding protein.

5. The purification container according to claim 1, wherein the target biomolecule is at least one member selected from the group consisting of a virus, a virus-like particle, a vaccine, an antibody, an antibody-like molecule, an antibody-binding protein, a hormone, a cytokine, a growth factor, an enzyme and a plasma protein.

6. The purification container according to claim 1, wherein a pore diameter of the filter is greater than or equal to 0.1 μm and less than or equal to 100 μm.

7. The purification container according to claim 1, further comprising a means for stirring the cell culture liquid or the body fluid and the adsorbent and/or the aggregating agent.

8. A method for purifying target biomolecule from a cell culture liquid or a body fluid, the method comprising the steps of:
supplying the cell culture liquid or the body fluid into the inlet of the purification container according to claim 1, and
contacting the cell culture liquid or the body fluid with the adsorbent and/or the aggregating agent.


SEQUENCE LISTING (2 sequences)

SEQ ID NO: 1 - DNA, 15 bp, Artificial Sequence
  Primer
gaggcgggcggatca

SEQ ID NO: 2 - DNA, 27 bp, Artificial Sequence
  Primer
cccggctaatttttgtatttttagtag